(19) United States
(12) Patent Application Publication
(10) Pub. No.: US 2004/0105849 A1
(43) Pub. Date: Jun. 3, 2004

(54) Treatment for SMA disease

(76) Inventor: Antonia C. Kaloidis, Woodbury, CT (US)

Correspondence Address: Thomas J. Monahan, Esq., Monahan & Costello, LLC, 4154 Madison Avenue, Trumbull, CT, 06611, US

(21) Appl. No.: 10/721,720

(22) Filed: Nov. 25, 2003

Related U.S. Application Data
(60) Provisional application No. 60/428,829, filed on Nov. 25, 2002.

(51) Int. Cl. A61K 45/00; A61K 31/7048; A61K 31/715; A61K 31/375; A61K 31/353

(52) U.S. Cl. 424/93.45; 514/27; 514/54; 514/456; 514/474

(57) ABSTRACT

There is provided compositions for the treatment of the genetic motor neuron disease Spinal Muscular Atrophy (SMA) and methods of using such compositions. The compositions preferably comprise vitamin C and at least two ingredients selected from the group consisting of: flavonoid, pectin, enteric bacterium, enzyme supplement, and any combinations thereof.

This application clams priority from U.S. Provisional Application No. 60/428,829 filed Nov. 25, 2002.

### BACKGROUND OF THE INVENTION

1. Field of the Invention

The present invention relates to medical treatment for Spinal Muscular Atrophy disease (hereinafter “SMA”). More particularly, the present invention relates to compositions and methods for their use in the treatment of SMA.

2. Description of the Prior Art

Spinal Muscular Atrophy (SMA) is a genetic motor neuron disease which is characterized by progressive degeneration of motor neurons in the spinal cord. More specifically, the disorder is characterized by skeletal muscle wasting due to progressive degeneration of anterior horn cells in the spinal cord and motor nuclei in the brain stem. Weakness is more often more severe in the legs than in the arms. SMA is an autosomal recessive disorder which requires both parents to be carriers of the gene responsible for the disorder and to pass genetic information onto their progeny. To be afflicted with SMA a child must have received the gene from each of its parents. Statistically, one in forty people are genetic carriers of SMA and one in every six to ten thousand live births is afflicted with this disease. Parents who carry the gene usually do not have symptoms of the disease. This frequency of occurrence is roughly comparable to that of amyotrophic lateral sclerosis, or Lou Gehrig's disease, which is a much more commonly known neuromuscular disorder. Even though both parents are carriers, the passing of the SMA causative gene along to a child occurs at a frequency of about 25%. The gene for SMA has been identified and diagnostic tests for the presence of this gene are available.

SMA is a genetic often fatal neuromuscular illness that causes loss of muscle control. It is primarily a childhood disease affecting the voluntary muscles for activities such as crawling, walking, head and neck control and swallowing. Leg weakness is usually more severe than arm weakness. Some individuals with SMA have abnormal movements of the tongue, know as tongue fasciculations. These symptoms are caused by a wasting away of nerve cells in the spinal cord which commonly leads to increasing muscular weakness, an inability to walk or stand, and in many cases death. Sometimes those with severe forms of SMA end up with collapsed lungs and must be kept alive with assisted breathing devices. SMA is one of the most common human genetic diseases. It is the leading genetic cause of death worldwide of children under two years of age. The senses, feelings and intellectual activity of those afflicted with SMA are usually normal. It is often observed that patients with SMA are unusually bright and sociable.

There are three main recognized variants of SMA. Acute SMA (Type I SMA; or Werdnig-Hoffmann disease) is the most severe form of this disease. Symptoms occur in infants by two to four months of age. All afflicted infants have delayed motor milestones by six months. There may be lack of fetal movement in the final months of pregnancy and most afflicted infants are hypotonic at birth. Diagnosis of the disease is usually made before six months and in most instances before three months of age. Even with early diagnosis, the disease does not necessarily follow the same course of severity for all children. Usually a child with Type I SMA is never able to lift his or her head or to reach normal physical milestones. Difficulty with swallowing, feeding and handling bodily secretions is common. Feeding and swallowing may be extremely difficult. Also, in Type I patients there is a general weakness in the intercostals and accessory muscles that are situated between the ribs. Due to reduced strength of the chest muscles resulting in diaphragmatic breathing or breathing in the abdominal areas, the chest may appear concave. In view of increasing overall weakness or repeated respiratory infections, the prognosis of children with Type I SMA is poor. Death occurs in the majority of children by two years of age.

The second common variant is Intermediate SMA (Type II SMA or Chronic SMA). Infants and children are symptomatic by age 2, and most at about six to twelve months. The majority of cases are diagnosed by fifteen months of age. Less than 25% of patients learn to sit and none learn to walk or crawl. Regardless of the age of the child at onset of the disease, Type II SMA children are hypotonic with flaccid muscle weakness, absent deep tendon reflexes, and tongue fsciculations that may be hard to notice in young children. Dysphagia may be present. Children with Type II SMA may be able to sit unsupported although they are usually unable to come to a sitting position without assistance. Sometimes children may be able to stand, but this is most often only possible with the aid of bracing and/or parapodium/standing frames. Feeding and swallowing problems are not usually characteristic of Type II SMA patients, but when present a feeding tube may be necessary. Fine tremors in outstretched fingers is common and most children with Type II SMA are diaphragmatic breathers. Although the disease is often fatal in early life, frequently from respiratory complications, there is a range of progression in patients with Type II SMA and it is hard to predict how fast, if at all, weakness will progress. Some children may lean to walk with the aid of bracing and survive into adulthood. However others with weakened chest and respiratory muscles may become increasingly feeble and susceptibility to respiratory infections such as pneumonia. In many cases the progression of the disease may stabilize, or there may be periods of disease progression followed by long periods of stability. In view of variable factors, the age of death from Type II SMA can vary widely occurring as early as three years of age or not until adulthood. Not all children with Type II SMA develop respiratory weakness. However, respiratory failure is usually the cause of death following respiratory infection such as pneumonia.

The third common variant of SMA is Mild SMA (Type III SMA; Wohlfart-Kugelberg-Welander disease; or Juvenile SMA) begins between two and seventeen years of age. It has similar pathologic findings and mode of inheritance to Types I and II SMA, but the disease is milder, has slower evolution of symptoms, and has a longer life expectancy. Weakness and muscle wasting are most evident in the legs, with onset in the quadriceps and hip flexors. Later, the arms are affected and weakness often progresses from proximal to distal parts. Some familial cases may be secondary to specific enzyme defects (e.g., hexosamimidase deficiency). Patients with Type III SMA can stand and walk, but may show difficulty with walking and/or getting up from sitting or bend over positions. Mild tremors can be seen in fingers, but tongue fasciculations are infrequent.

In addition to Types I, II and II SMA, there are two less frequently occurring variants: (i) Adult Onset SMA (Type IV SMA); and (ii) Adult Onset X-linked SMA. With Type IV SMA, symptoms begin in adults after 35 years of age. It is very rare for SMA to begin between 18 and 30 years of age. Type IV SMA is characterized by insidious onset and very slow disease progression. The muscles used for swallowing and respiratory functions (bulbar muscles) are rarely affected in Type IV SMA patients.

Adult Onset X-Linked SMA is also known as Kennedy's Syndrome or Bulbo-Spinal Muscular Atrophy. It occurs only in males, although 50% of female offspring are carriers. This form of SMA is associated with a mutation in the gene that codes for part of the androgen receptor and male patents often have breast enlargement known as gynecomastia. Facial and tongue muscles are noticeable affected. The course of Adult Onset X-liked SMA is variable, but generally tends to be slowly progressive or stationary.

The neuromuscular disease SMA, which leads to degeneration of motor neurons of the spinal cord and associated muscular weakness and atrophy, is caused by deletions or mutations of one or two of the genes [e.g., the survival motor neuron gene(SMN)] that code for the survival of motor neuron or SMN protein. The survival motor neuron gene (SMN) is present as an inverted repeat on chromosome 5 at 5q13, and over 98% of children with SMA have deletions or mutations of the telomeric copy of the gene (SMN1) resulting in reduced levels of SMN protein (Friesen et al., Mol. Cell, 7: 1111-1117, 2001). In addition to the gene SMN1, most people have several copies of the backup gene SMN2 that produces a much smaller amount of SMN protein. It is believed that the copy number of SMN2 genes determines the severity of SMA disease. As higher copy numbers of SMN2 impact patient survival, development of a drug that could stimulate increased activity of SMN2 to produce more SMN protein would be useful. Research to manipulate the SMN2 gene are underway in the United States and Europe and preliminary results from cell culture studies are encouraging. However, as with any drug approved by the U.S. Food and Drug Administration, several years of testing and clinical trails would be required before any drug for the treatment of SMA could reach the market. There is a long-felt need for treatment of SMA, especially in view of the fact that currently death by the age of two is almost certain for children with Type I SMA.

SMN protein is found in all animals that have bodies composed of cells that have differentiated into tissues and organs and is required for the normal functioning of motor neurons. The absence or reduced levels of this protein is believed to trigger the common, often lethal, motor neuron degenerative SMA disease. The explanation for the reason why low levels of SMN give rise to the destruction of motor neurons remains to be elucidated. Motor neurons are specifically lost and other cell types are unaffected. Deletion of the gene(s) that code for SMN protein results in a defect or a reduction of the SMN protein in the body. In effect, SMA is the disease that comprises the lack of normal SMN. When the appropriate level of SMN protein is lacking, the nerve cells that serve major muscle groups are damaged. This is followed by wasting of the muscles due to a lack of stimulation.

The biochemical activity of the survival of motor neurons protein (SMN) was recently investigated and the importance of the methylation state of proteins that interact with the SMN complex was demonstrated (Friesen, W. J., 2001, supra). It was reported that SMN only interacts with its substrates after they are modified to dimethyarginines (Friesen, W. J., et al., 2001, supra, p. 1114). It is believed that the SMN protein binds to “target” proteins in the cell and that SMN binds to these target proteins only when the arginine residues have been dimethylated. This allows for a more “snug” fit into the target protein site and enables the SMN protein to function appropriately. These SMN target proteins obtain their methyl groups from a methyl donor called S-adenylmethionine, which itself depends on folate and vitamin B₁₂ as part of its metabolic pathway. The key role of methylation in protein interaction with SMN suggests that deficiencies in this methylation would have similar consequences to reduced levels of, or mutations in SMN, as is the case in SMA disease. It was suggested that undermethylation of proteins would further aggravate the severity of SMA and that it may be advisable to provide dietary supplementation to SMA patients with factors that contribute to an optimal methylation state, including folic acid, vitamin B₁₂ and vitamin B₆. It is generally known that the nervous system is particularly sensitive to deficiencies in folic acid and vitamin B₁₂ (Friesen, W. J., et al., 2001, supra, p. 1115).

Folic acid and vitamin B₁₂ cannot either be produced or stored but must be obtained through dietary intake and there is evidence that the severity of SMA may be ameliorated by these common vitamins. The results of Friesen et al. raise the possibility that deficiencies in folic acid or the B vitamins could be detrimental to SMA patients and result in under methylation of proteins which are required for SMN to function properly. Many vegetables, grains and fruits are rich sources of folic acid. The recommended daily allowances for B₁₂ and folic acid for children and adults are known and are generally available in a regular balanced diet. Most infant formulas are supplemented with both of these vitamins.

It is known that bacteria located in the large intestine synthesize B-vitamins. The exact quantities of vitamins that are synthesized endogenously, as well as their impact on methylation, has not been determined. However, it is known that individuals who have been given the antibiotic trimethoprim-sulfamethoxasole for extended periods of time may develop a folic acid deficiency anemia due to bacterial changes as a result of a reduction in the B-vitamin producing bacteria in the large bowel. It is understood that inducing a change in bowel flora may result in a subsequent decrease in folic acid levels in the body.

Perhaps, research should be directed towards what causes a drastic reduction in the co-factors (B12 and folate) necessary for the “snug fit” of SMN into the target protein site. Perhaps the decline in SMN levels is due to a mutation of the protein as a result of its inability to fit into the target protein site. Consequently, the rate limiting step in the equation then becomes the body's inability to provide an appropriate methylated target site for the SMN. If this is indeed the case, then further increasing SMN levels in these individuals will only provide temporary relief and valuable time will be lost.

U.S. Pat. No. 6,376,508 to Li et al. and U.S. Patent Application Publication No. 2003/0040543 are directed to treatments for spinal muscular atrophy. However, in spite of such proposals and the promise of future treatments for SMA arising from ongoing research programs, there is currently no cure or medical treatment for this disease. Only palliative care can now be offered to patients. There is no known therapy to reverse the course of SMA.

Accordingly, it is an object of the present invention to provide compositions for the treatment of spinal muscular atrophy.

Another object of the present invention is to provide a new and improved method for the treatment of spinal muscular atrophy.

### SUMMARY OF THE INVENTION

The present invention is directed to novel methods of treating the effects of the genetic motor neuron disease Spinal Muscular Atrophy (SMA). The method of the invention comprises administering a pharmacologically effective and physiologically acceptable amount of vitamin C and at least one ingredient selected from the group consisting of: flavonoid, pectin, enteric bacterium, enzyme supplement and any combinations thereof; and optionally, a pharmaceutically acceptable carrier. The preferred flavonoid is citrus bioflavonoid. The flavonoid preferably has at least one flavonoid ingredient selected from the group consisting of: rutin, hesperidin, naringin, naringenin 7-B rutinoside, flavonols, quercetin, flavones, phenolics, and any combinations thereof. The pectin ingredient may be citrus pectin, pomace pectin, and any combinations thereof. The preferred pectin is apple pectin. The enteric bacterium ingredient comprises at least one bacterium selected from the group consisting of Lactobacillus acidophilus, Bifidobacterium bifidum, Lactobacillus rhamnosus, Enterococcus faecium, Bifidobacterium adolescentis, Lactobacillus plantrum, and any combinations thereof. A particularly preferred combination for treating SMA is citrus flavonoid and apple pectin and enteric bacterial supplement. The enzyme supplement ingredient has at least one enzyme selected from the group consisting of: amylase, protease, lactase, cellulose, lipase, phytase, sucrase, maltase, and any combinations thereof. The optional pharmaceutically acceptable carrier is a solid or liquid selected from the group of water, aqueous systems, alcohols, polyols, glycols, mineral oils, vegetable oils, excipient, binder and any combinations thereof. The ingredients and the optional pharmaceutically acceptable carrier may be in a product form selected from the group consisting of an aerosol spray, pump spray, cream, emulsion, solid, liquid, dispersion, foam, gel and powder.

Compositions for the treatment of the effects of the genetic motor neuron disease Spinal Muscular Atrophy (SMA) in humans comprise: vitamin C; at least two ingredients selected from the group consisting of: flavonoid, pectin, at least one enteric bacterium, enzyme supplement, and any combinations thereof; and an optional pharmaceutically acceptable carrier. The composition of the invention has vitamin C and at least two ingredients in pharmacologically and physiologically acceptable amounts sufficient to mitigate the symptoms of SMA. The flavonoid ingredient of the composition comprises at least one flavonoid selected from the group consisting of: rutin, hesperidin, quercetin, naringin, naringenin 7-B rutinoside, flavonols, flavones, phenolics, and any combinations thereof. The pectin of the composition may be citrus pectin, pomace pectin, and any combinations thereof. However, apple pectin is preferred. The enzyme supplement ingredient of the composition has at least one enzyme selected from the group consisting of: amylase, protease, lactase, cellulose, lipase, phytase, sucrase, maltase, and any combinations thereof. The optional pharmaceutically acceptable carrier may be a solid or liquid selected from the group of water, aqueous systems, alcohols, polyols, glycols, mineral oils, vegetable oils, excipient, binder, powder and any combinations thereof. The preferred optional pharmaceutically acceptable carrier is water. The compositions of the invention may be in a product form selected from the group consisting of an aerosol spray, pump spray, cream, emulsion, solid, liquid, dispersion, foam, gel and powder. The compositions may contain at least one bacterium selected from the group consisting of Lactobacillus acidophilus, Bifidobacterium bifidum, Lactobacillus rhamnosus, Enterococcus faecium, Bifidobacterium adolescentis, Lactobacillus plantrum, and any combinations thereof.

Unless otherwise defined, all technical and scientific terms used herein have the same meaning as commonly understood by one of ordinary skill in the art to which this invention belongs. Although methods and materials similar or equivalent to those described herein can be used in the practice or testing of the present invention, the preferred methods and materials are described. All publications, patent applications, patent and any other references mentioned herein are incorporated by reference in their entirety. In case of conflict, the present specification, including definitions, will control. In addition, the materials, methods and examples are illustrative only and not intended to be limiting.

Other features and advantages of the invention will be apparent from the following detailed description and from the claims.

### DETAILED DESCRIPTION OF THE INVENTION

The present invention is directed to compositions and a novel method for the treatment of SMA disease. Although the present invention suggests various mechanisms for the experimental results described herein, the exact mechanisms involved remains to be completely elucidated and the invention is not limited thereby. Consideration of the role of enteric microorganism is a foundation of the present invention. Enteric microorganisms are a large group of microbes whose natural habitat is the intestinal tract of man and animals. They are largely gram-negative, non-spore forming rods. Some of these microorganisms (e.g., Escherichia coli and Aerobacter aerogenes) form part of the normal flora of the intestinal tract while others (e.g., Salmonellae, Shigellae) are regularly pathogenic to man.

It is suggested that perhaps additional genetic factors that are currently unknown may be involved in individuals with SMA and that these code for a structural abnormality in the large intestine. These deletions may render the large bowel inhospitable to the attachment and proliferation of life sustaining enteric or gut bacteria. Although considerable variation in the composition of intestinal flora can be found among individuals, studies show that it remains quite stable in a single individual over time. Thus, it is the environment of the large bowel (under genetic control) which determines which bacteria will colonize. Studies using identical twins support the generalization that the composition and function of microbes in the intestines is environmentally controlled.

If this presumptively abnormal intestinal flora of SMA patients was to be reversed, and normal life sustaining bacteria were to adhere and colonize the large intestine then (i) ample supplies of B₁₂ and folate would be available for the methylation of proteins which would facilitate the normal functioning of the SMN protein; (ii) acidification of the intestinal environment would result in an increased absorption of calcium, zinc, iron and copper; and (iii) there would be an increase in the deconjugation of bile acids and promotion of enterohepatic circulation resulting in an increase in the absorption of fats and fat soluble vitamins. It is suggested that believe that these three factors are involved in the overall disease process of SMA. The present invention provides a solution to the critical need for treatment of the SMA condition.

Normally, bacteria in human large intestines perform the function of releasing B-vitamins in a low dose “sustained release” fashion. Also, enteric bacteria are responsible for the recirculation of bile salts. Without enough bile, SMA patients are not able to properly absorb fat-soluble vitamins. In addition, intestinal bacteria are also responsible for the proper assimilation of calcium and other minerals.

The biologic role of the intestinal flora includes the following: (i) barrier protection against colonization of pathogens; (ii) regulation of intestinal transit; (iii) deconjugation of bile acids and promotion of enterohepatic circulation; (iv) degradation and digestion of some undigested carbohydrates; (v) improvement of lactose intolerance; (vi) production of vitamins and pre-protein digestion; and (vii) local production of short chain fatty acids directly or indirectly which contributes to the regulation of water and electrolyte absorption in the large bowel.

The present invention discloses treating SMA disease using a preferable combination of vitamin C (ascorbic acid) and nutrients, preferably but not limited to bioflavonoids (preferably but not limited to citrus bioflavonoids), and pectin (preferably but not limited to apple pectin) and probiotics (enteric bacteria) that interact and enhance bacterial colonization of the large bowel. Use of these compositions helps to facilitate and maintain attachment of life sustaining bacteria to the epithelial layer of the large intestine.

Early Symptoms and Patient Diagnosis. The patient (“Patient A”), a Caucasian female, was born on Sep. 20, 1996. She was normal at birth with no signs of muscular weakness. Within three months of her birth, she developed a skin condition. She had a very severe case of Infantile Seborrhoeic Dermatitis which lasted for 8-9 months. Infantile Seborrhoeic Dermatitis, also known as cradle cap, is a condition that occurs in infants and is caused by the drying of excess oils excreted from the infant's body. It is characterized by a yellowish-whitish crust on the infant's head. Following medical consultation, various topical creams and ointments were employed without success. The B vitamin biotin was then given at a dosage of about 4,000 mcg at intervals of three days per week. Current research attributes cradle cap to a deficiency of this vitamin. The patient's cradle cap resolved itself almost immediately. Gut bacteria located in the large intestine are responsible for the production this B vitamin and a deficiency of this nutrient under normal conditions is rare.

The patient then began developing a protruding belly and “bumpy skin” on the back of her arms, much like children who are vitamin A and protein deficient. This was suggestive of some sort of a malabsorption syndrome. By 6 months of age, the patient was unable to sit and had lost the ability to hold her head up. Her breathing was becoming labored and she would sweat profusely. While she was aware of her environment, her ability to interact with her toys and sibling was greatly diminishing. It appeared that she was decompensating rapidly. In July 1997, she was diagnosed with Spinal Muscular Atrophy (SMA) Type I at Connecticut Children's Medical Center in Hartford, Conn. Physicians advised that there was no cure for SMA and that palliative measures could be instituted. Because of the gravity of her condition, the medical prognosis was grave and death was predicted to statistically occur within three to six months. There was no experience of ever seeing a remission in a patient with severe Type I SMA and death was likely to occur within three months.

Administration of Topical And Oral Vitamins. Two questions regarding the development of a treatment for SMA were considered. Firstly, why was Patient A born apparently “normal” and then began deteriorating with symptoms of SMA? Secondly, why is there such a wide spectrum of disability in SMA? It was postulated that if Patient A could produce adequate SMN for three months after birth, then why couldn't it be possible to have her produce appropriate levels of SMN later? What made some children afflicted with SMA lose their ability to produce SMN early in life, while others were able to produce enough SMN to sustain life until various ages? The focus was on SMA type I of Patient A and acquiring an understanding of what was happening during the course of the disease. If the hypothesis was correct for Type I SMA, then it could be extrapolated to perhaps explain all types of SMA. It was also possible that SMA Type I was a distinctly different disease from the other known SMA variants.

It was possible that in utero nutrition was delivered via the umbilicus during gestation of Patient A. Perhaps this type of delivery of nutrition into the body enabled SMA Patient A to obtain the nutrition necessary to produce an adequate amount of appropriately functioning SMN. If the disease was a disease of malabsorption, then the problem would involve the large and/or small intestine. The digestive process was reviewed in detail for a digestive enzyme deficiency that would produce the symptoms shown in Patient A. This approach was not believed correct as, if indeed an enzyme or digestive constituent was missing, then the symptomology and course of the disease would be quite definable with a definite set of symptoms, life expectancy etc. which was not the case. If all the necessary enzymes and constituents of digestion were present, then perhaps there might be a structural abnormality responsible for Patient A's condition. Perhaps the epithelial layer was abnormal or intestinal villi were too short. SMA children could possibly born with varying degrees of such structural abnormality.

Believing that Patient A was not absorbing nutrients, especially fats, transdermal administration of nutrients was considered. The condition of Patient A was worsening. Fish and Evening Primrose oils were applied all over the body of Patient A. Vitamins A and E were then applied, and Patient A's torso was wrapped in Saran wrap to facilitate transdermal absorption. The dosage was 5,000 IU of Vitamin A and 400 IU of Vitamin E, once daily for a time period of approximately 8 hours. Patient A's respiratory function would improve for a short period of time. Her breathing would be less labored and her coloration improved (i.e., became less gray).

The administration of oral vitamins, especially fat soluble vitamins gave a poor response. Patient A would turn pale and her respiration would increase. She would breathe quickly and in an ineffective manner.

The administration of topical vitamins was continued. The challenge was to get effective low doses of B vitamins and the fat-soluble vitamins into her body at a steady rate. It was hoped that more consistent results could be obtained with respect to her respiratory status and her ability for spontaneous movement. She appeared to improve when vitamins were administered. However, this improvement was short lived and erratic. Topical, sub-lingual, and even aerosol dosage forms were tried, all with the same results. The physiology of the small intestine was considered. It seemed as though some of Patient A's symptoms correlated with a deficiency of folic acid. Focus was then put on the metabolism and transport of folic acid across the intestinal lumen. Various “activated” forms of folic acid were tried with no sustainable results.

It is known that when the gut flora is modified and contains an overgrowth of pathogenic bacteria and Candida, then vitamin supplements cannot be metabolized appropriately. Some patients experience an almost “toxic” response to supplements until the balance of healthy flora is restored in their lower intestine. Modification of Patient A's lower intestinal environment would be required followed by administration of vitamin supplements. Research then focused on the anatomy and physiology of the large intestine and how to repopulate Patient A's intestine with life sustaining bacteria.

The issue of folic acid assimilation in the small intestine was deferred until Patient A became stronger. In January, 1998, at 1 year and 4 months of age, probiotic bacteria were administered to Patient A.

Repopulation of Intestinal Bacteria. A stool sample from Patient A was tested by Great Smokies Diagnostic labs. The results indicated that Patient A had intestinal dysbiosis with an overgrowth of an assortment of pathogenic bacteria and Candida. While long chain fatty acids, cholesterol and total fecal fats were all within reference range, total short chain fatty acids were depressed. Stool pH was elevated and butyrate depressed. Distribution of short chain fatty acids was imbalanced.

Supplemental bacteria were administered to Patient A to rebalance her intestinal flora. Supplemental bacteria would have to be able to: (1) attach to the epithelial layer, (2) reproduce and flourish in the human body and (3) survive in Patient A's large intestinal environment. With these prerequisites in mind, various strains of bacteria were tried, including even home made yogurt, in an effort to repopulate Patient A's intestine. Over a period of about one year, various strains of bacteria were studied in an attempt to identify exactly which types would populate Patient A's large intestine. A temporary improvement in Patient A's condition was obtained which then returned back to her baseline condition. An enterogenic concentrate manufactured by Tyler Encapsulations was administered to Patient A. This product contains Lactobacillus acidophilus, Bifidobacteria Bifidum, Bifidobacteria infantis, Enterococcus faecium and fructoolegosaccharides. A dosage of 1 capsule one day per week was used.

Administration of Citrus Bioflavonoids. Patient A had just turned two years old and she was having great difficulty breathing. An objective of the treatment of the patient was to decrease the mucus production in her lungs and enhance her ability to breathe. Citrus bioflavonoids have antihistaminic properties. They can, for example, be taken for the treatment of asthma. The Thorn Research brand of Vitamin C with bioflavonoids was selected and administered to Patient A in September 1998. This product contains 500 mg of Vitamin C and 75 mg of bioflavonoids, which was administered at a dose rate of ½ capsule three days per week. The total dose of bioflavoioids was 35 mg three days per week.

In November SMA Patient A was afflicted with the flu. It developed into pneumonia and she was intubated and placed on a respirator. It was predicted that there was no chance that she could be weaned from the respirator. To the great surprise of her physicians, Patient A was weaned and was able to breathe without the aid of BiPAP. Genetic testing was then repeated to ascertain that she indeed did have SMA. SMA was confirmed. Patient A was continued on citrus bioflavonoids three times weekly along with the Enterogenic Concentrate. Using an oximeter it was noted that her oxygen saturations would increase after the administration of bioflavonoids. A decrease in the amount of mucus in her lungs was also observed. The dosage was Thorne Research Vitamin C with bioflavoioids, administered at a dose of ½ capsule three days per week. The Enterogenic Concentrate was administered at a rate of one capsule one day per week.

The effects on the body of bioflavonoids on the body were studied. It has been reported that citrus bioflavonoids have an effect on capillary fragility and allergies. Although there was no specific scientific reason to continue giving bioflavenoids to Patient A, the administration of bioflavonoids was continued. It was then realized that the bioflavonoids played a significant role in the treatment of SMA. They seemed to increase oxygen saturations for about 12-24 hours. When given on a daily basis however, her body could not tolerate the effects. She would become pale and lethargic.

Flavonoid pigments are a group or series of related water soluble phenolilc glycosides having a common basic structural unit (i.e, the C₁₅ skeleton of flavone). The flavone carbon structure consists of two C₆ groups (substituted benzene rings) connected by a three carbon aliphatic chain. There are, for example, the scarlet, crimson and purple anthocyanins (e.g., cyanidin 3-rutinoside), which, other than the chlorophylls, are the most important group of coloring materials in higher plants. Also, there are the ivory or pale yellow flavones (i.e., rutin) which occur as widely as the anthocyanins but which contribute more modestly to plant color. There are flavonoids of less frequent occurrence, such as the yellow chalcones and aurones and the colorless flavanones and isoflavanones. Flavonoids occur almost universally in higher plants. They are not synthesized by animals. However, some are known to be physiologically important in animals. Perhaps the only undisputed function of flavonoids in plants is their contribution to flower and fruit color.

The Administration of Quercetin. In May 2001, Patient A was given quercetin. Quercitin is a potent antihistamine. Quercitin (300 mg), Scientific Botanicals brand, was given at a dose rate of ½ capsule three days per week. The substrate for dividing bacteria is supplied by the constant production of fresh mucin by mucosal goblet cells in the large intestine. Mucin carbohydrate can be fermented by the anaerobes of Bifidobacteria species. It was postulated that a lack of these bacteria in the large intestine would allow for the mucin layer to be over produced without any regulation. Patient A had mucus in her stool as well as in her lungs and nasal passage. The mucus was copious and stringy in form. Quercitin was used to dry up some of the mucus. However, the dosage had to be carefully regulated as too much quercetin would depress Patient A's respiration. The quercetin was supplemented until April, 2002 when it was no longer needed. It served as a supportive agent in the event she become ill.

Changing Bioflavonoid Brand; Changing Supplemental Bacteria Brand. In May 2002, Patient A was given Kyo-Dophilus. This product contains a combination of three bacteria: Lactobacillus acidophilus, Bifidobacteria bifidus and Bifidobacteria Iongum. This product is produced by the Japanese company Wakunaga of America.

Citrus bioflavonoids are extracted from the rind of citrus fruits. There are limitations regarding products containing bioflavonoid components. As a nutraceutical product, there is no industry standard for citrus bioflavonoids. Formulations vary with respect to types of fruit used and the active ingredients present. Various different brands of bioflavonoids were tested and given to Patient A. In October 2002, the AMNI Brand of Ester C with bioflavonoids was given in an effort to obtain even better results.

Assesment Parameters. Various parameters may be used to determine treatment efficacy, including but not limited to, the following: (i) endurance for daily activities; (ii) decrease in nasal, chest and bowel mucus; (iii) Increase in spontaneous movement; and (iii) increase in oxygen saturations.

Administration of Apple Pectin; Rutin. In September, 2002, Patient A's stool was analyzed by Great Smokies Diagnostic Labs. While the results showed a moderate amount of Lactobacillus Acidophilus to be present, Bifidobacteria cultures produced no growth. Apple pectin was then added to her regimen. It was hoped that this soluble fiber (found in apple peel) would increase short chain fatty acid synthesis and lower intestinal pH. It was also hoped that pectin would stimulate the growth of Bifidobacteria in the large intestine. Although apple pectin is a preferred embodiment of the present invention, any convenient source of pectin may be utilized (i.e., pectin from citrus or the pomaces, or combinations).

Pectins are polysaccharide substances (mol. Wt. 20,000-400,000) present in the cell walls of all plant tissues which function as an intercellular cementing material. One of the richest sources of pectin is lemon or orange rind which contains about 30% of this polysaccharide. Pectin occurs as a course or fine powder, yellowish-white in color, practically odorless, and with a mucilaginous taste. It is almost completely soluble in 20 parts water. Pectin is derived commercially by dilute acid extraction of the inner portion of the rind of citrus fruits, or the fruits of pomaces, usually apple. A common characteristic of the pectins is their ability to jell at room temperature, typically after addition of sugar and fruit juices in the preparation of jams or jellies.

Rutin is an ingredient of pectin. Addition of rutin might serve as a substitute for pectin, but addition of rutin along with pectin did not appear to produce any added benefit to Patient A and caused her lips to blister and peel. Pectin without rutin supplementation is preferred. Rutin and hesperidin are part of the vitamin P group and function synergistically with vitamin C. It appears that Rutin may have some anticholinergic activity. This enhances gut motility and encourages the growth of appropriate bacteria in the lower gut.

Rutin (Freeda Vitamins, Inc., 36 East 41st Street, New York, N.Y. 10017) as a capsule serving size of 50 mg. The dosage used to treat Patient A was approximately 3 mg every three days. Dosages below this level are ineffective and higher dosages cause nausea, decrease gastrointestinal motility and drowsiness.

Digestive Enzyme Supplementation. The provision of supplemental enzymes may be beneficial. Digestive enzymes may optionally be given to enable the breakdown and assimilation of food and nutrients during the digestive process. There is evidence that enhancing the intestinal breakdown of protein fosters the growth of healthy bacteria in the intestine. Commercial enzyme supplements (i.e., Similase Jr.) supply lactase and other enzymes that are normally produced by enteric bacteria. Enzyme supplements may also have the effect of further acidifying the large bowel. Enzyme supplements may contain, but are not limited to: amylase, protease, lactase, cellulase, lipase, phytase, sucrase and maltase. Similase Jr. may optionally be administered at a dose rate of about ½ capsule every three days as greater dosages cause constipation.

Changing brand of citrus bioflavonoids; In early 2003, the condition of Patient A was deteriorating. Her arms were becoming weaker and she was becoming ill more often. In April, 2003 she contracted S. pneumonia and was intubated for a period of 13 days. An attempt to extubate was made on day 7 and she had to be reintubated. Permanent ventilation was being considered. There was concern as to why Patient A appeared to be deteriorating so quickly. Her deterioration coincided with a change in brand of bioflavonoid that was made in December, 2002. Patient A was changed to the Vital Nutrients brand bioflavonoid supplement. Administering 1 capsule (500 mg vitamin C/250 mg bioflavonoids) dissolved in water gave the patient approximately 75 mg of bioflavonoids every hour until she began to breathe with greater ease and clear mucus more effectively.

In October, 2003 Culturelle was added to the probiotic regimen. It was postulated that this particular probiotic would contribute significantly to the acidification of the large bowel. Lactobacillus rhamnosus GG has been shown to increase the level of total anaerobic flora, especially Bifidobacteria, Bacteroides and Clostridia. Preferred dose of Culturelle is 1 capsule once a week for Patient A. Higher doses produce stomach upset and diarrhea.

Patient A remains on the above supplements (Culturelle, Kyo-Dophlus, Vital Nutients Vitamin C with Bioflavonoids, and Twin Laborabories Apple Pectin). The Vitamin C with Bioflavonoids, Pectin, and Probiotics can conveniently be administered together. They are dissolved in distilled water and injected into patient A's gastric feeding tube. The ingredients are in powder form and may also be administered to SMA patients in the original capsule form.

Vitamin C with Bioflavonoids/Apple Pectin/Kyo-Dophilus/Culturelle. Vital Nutrients brand of Vitamin Complex with bioflavonoids, Twin Lab brand of apple pectin, Kyo-Dophilus, and Culturelle are preferred ingredients for the treatment of SMA. Patient A remains on this combination of ingredients. It is understood that commercial sources of individual ingredients used in the present invention for the treatment of SMA may contain various excipients known in the art. Excipients are generally inert substances (i.e., silica, cellulose, starch, gum arabic etc) added to active ingredients to form a suitable vehicle for administration.

The preferred ingredients used for treatment of SMA may conveniently be administered in concert or individually at the dosage and time course indicated for the treatment of Patient A, a 19 Kg female with Type I SMA, the most severe variant of this disease. It is understood that the amounts of the various ingredients of the method of the invention can be, as may be necessary, conveniently be adjusted up or down for body weigh and other variants of the disease. The mode of delivery may vary. For example, the delivery to Patient A was preferably by means of a gastric tube with distilled water solubilized ingredients. Ingredients could also be injected into the gastric tube in solid, gel or semi-solid form. Other children may swallow ingredients in solid or liquid form (with pharmaceutically acceptable carriers, e.g., water).

The citrus bioflavonoid/apple pectin/probiotic combination appears to modify the intestinal environment in one or more of the following ways: (i) they facilitate the adherence of the bacteria to the cell wall; (ii) they affect a change in the environment of the large intestine, perhaps decreasing intestinal pH; and (iii) they are used as a fermentation product by the bacteria of the large intestine possibly increasing production of short chain fatty acids and/or decreasing intestinal pH; (iv) impacting gastrointestinal motility.

The serving size of Vitamin C with Bioflavonoids is one capsule (Vital Nutrients/RHG and Co., Inc., Middletown, Conn. 06457). Each capsule contains 500 mg of vitamin C (100% Ascorbic Acid) and 250 mg of Citrus Bioflavonoid (60% complex). The total flavonoid composition has: 41.4% Hesperidin; 17.4% Naringin, Naringenin 7-B Rutinoside; and 1.2% Flavonols, Flavones and Phenolics. The preferred dosage for Patient A was 150 mg Vitamin C/75 mg bioflavonoids (¼ capsule) to be taken every three days. A dosage of 75 mg Vitamin C/35 mg bioflavonoids (⅛ capsule) was found to be too low and does not produce benefit. A dosage of 300 mg Vitamin C/250 mg bioflavonoids (½ capsule) was found to be too high and caused constipation.

Apple Pectin (Twin Laboratories, Ronkonkoma, N.Y. 11779) having a serving size of 1 capsule (500 mg USP) was used to treat Patient A. The preferred dosage for Patient A was 125 mg (¼ capsule) every 3 days. A dosage rate of 75 mg every 3 days was found to be ineffective and dosage of 250 mg caused constipation, bloating and a decrease in gastrointestinal motility.

Kyo-Dophilus probiotic supplement (Wakanaga of America) is a preferred enteric bacterial supplement. The preferred dose for patient A is 1 capsule once a week. Higher doses produced stomach upset and constipation with Patient A. Alternative probiotic supplements contain additional bacteria (i.e., Lactobacillus rhamnosus, Enterococcus faecium, Bifidobacterium adolescentis, and lactobacillus plantarum).

Culturelle is manufactured by CAG Functional Foods. P.O. Box 2820, Omaha, Nebr. 68103-0820. The preferred dose for patient A is 1 capsule weekly. Higher doses produce diarrhea.

Although the present invention describes in detail certain embodiments, it is understood that variations and modifications exist known to those skilled in the art that are within the invention. Accordingly, the present invention is intended to encompass all such alternatives, modifications and variations that are within the scope of the invention as set for in the following claims.

What is claimed is:

1. A method of treating the effects of the genetic motor neuron disease Spinal Muscular Atrophy (SMA) comprising:
administering a pharmacologically effective and physiologically acceptable amount of vitamin C and at least one ingredient selected from the group consisting of: flavonoid, pectin, enteric bacterium, enzyme supplement and any combinations thereof; and optionally, a pharmaceutically acceptable carrier.

2. The method of claim 1, wherein said flavonoid is citrus flavonoid.

3. The method of claim 1, wherein said flavonoid comprises at least one flavonoid ingredient selected from the group consisting of: rutin, hesperidin, naringin, naringenin 7-B rutinoside, flavonols, quercetin, flavones, phenolics, and any combinations thereof.

4. The method of claim 1, wherein said pectin is selected from the group consisting of: citrus pectin, pomace pectin, and any combinations thereof.

5. The method of claim 1, wherein said pectin is apple pectin

6. The method of claim 1, wherein said enteric bacterium comprises at least one bacterium selected from the group consisting of Lactobacillus acidophilus, Bifidobacterium bifidum, Lactobacillus rhamnosus, Enterococcus faecium, Bifidobacterium adolescentis, Lactobacillus plantrum, and any combinations thereof.

7. The method of claim 1, wherein said flavonoid is citrus flavonoid and said pectin is apple pectin.

8. The method of claim 1, wherein said at least one ingredient is flavoloid, pectin and enteric bacterium.

9. The method of claim 1, wherein said flavonoid is at least one citrus flavonoid selected from the group consisting of: rutin, hesperidin, naringin, naringenin 7-B rutinoside, flavonols, flavones, phenolics, and any combinations thereof; said pectin is at least one pectin selected from the group consisting of citrus pectin, pomace pectin, and any combination thereof; and said enteric bacterium is at least one bacterium selected from the group consisting of Lactobacillus acidophilus, Bifidobacterium bifidum, Lactobacillus rhamnosus, Enterococcus faecium, Bifidobactedum adolescentis, Lactobacillus plantrum, and any combinations thereof.

10. The method of claim 1, wherein said enzyme supplement is at least one enzyme selected from the group consisting of: amylase, protease, lactase, cellulose, lipase, phytase, sucrase, maltase, and any combinations thereof

11. The method of claim 1, wherein said optional pharmaceutically acceptable carrier is a solid or liquid selected from the group of water, aqueous systems, alcohols, polyols, glycols, mineral oils, vegetable oils, excipient, binder and any combinations thereof.

12. The method of claim 1, wherein said vitamin C, said at least one ingredient, and said optional pharmaceutically acceptable carrier are in a product form selected from the group consisting of an aerosol spray, pump spray, cream, emulsion, solid, liquid, dispersion, foam, gel and powder.

13. A composition for the treatment of the effects of the genetic motor neuron disease Spinal Muscular Atrophy (SMA) in humans comprising:
vitamin C;
at least two ingredients selected from the group consisting of: flavonoid, pectin, at least one enteric bacterium, enzyme supplement, and any combinations thereof; and
an optional pharmaceutically acceptable carrier.

14. The composition of claim 13, wherein said vitamin C and said at least two ingredients are in pharmacologically and physiologically acceptable amounts sufficient to mitigate the symptoms of SMA.

15. The composition of claim 13, wherein said flavonoid comprises at least one flavonoid selected from the group consisting of: rutin, hesperidin, quercetin, naringin, naringenin 7-B rutinoside, flavonols, flavones, phenolics, and any combinations thereof.

16. The composition of claim 13, wherein said pectin is selected from the group of citrus pectin, pomace pectin, and any combinations thereof.

17. The composition of claim 13, wherein said pectin is apple pectin.

18. The composition of claim 13, wherein said optional pharmaceutically acceptable carrier is a solid or liquid selected from the group of water, aqueous systems, alcohols, polyols, glycols, mineral oils, vegetable oils, excipient, binder, powder and any combinations thereof.

19. The composition of claim 13, wherein said vitamin C, said at least one ingredient, and said optional pharmaceutically acceptable carrier are in a product form selected from the group consisting of an aerosol spray, pump spray, cream, emulsion, solid, liquid, dispersion, foam, gel and powder.

20. The composition of claim 13, wherein said at least one bacterium is selected from the group consisting of Lactobacillus acidophilus, Bifidobacterium bifidum, Lactobacillus rhamnosus, Enterococcus faecium, Bifidobacterium adolescentis, Lactobacillus plantrum, and any combinations thereof.

21. The composition of claim 13, wherein said at least one ingredient is flavonoid and pectin, and said optional pharmaceutically acceptable carrier is water or aqueous system.

22. The composition of claim 13, wherein said at least one ingredient is flavonoids, pectin and at least one enteric bacterium, and said optional pharmaceutically acceptable optional carrier is a solid or liquid selected from the group of water, aqueous systems, alcohols, polyols, glycols, mineral oils, vegetable oils, excipient, binder, powder and any combinations thereof.

23. The composition of claim 13, wherein said flavonoid is citrus flavonoid, said pectin is pomace pectin, and said at least one bacterium comprises a bacterium selected from the group consisting of Lactobacillus acidophilus, Bifidobacterium bifidum, Lactobacillus rhamnosus, Enterococcus faecium, Bifidobacterium adolescentis, Lactobacillus plantrum, and any combinations thereof.

24. The composition of claim 13, wherein said flavonoid comprises at least one flavonoid selected from the group consisting of: rutin, hesperidin, naringin, naringenin 7-B rutinoside, flavonols, flavones, phenolics, and any combinations thereof, said pectin is apple pectin, and said at least one bacterium comprises a bacterium selected from the group consisting of Lactobacillus acidophilus, Bifidobacterium bifidum, Lactobacillus rhamnosus, Enterococcus faecium, Bifidobacterium adolescentis, Lactobacillus plantrum, and any combinations thereof.
